SEQUENCE LISTING (8 sequences)

SEQ ID NO: 1 - DNA, 195 bp, Homo sapiens
  source 1..195: mol_type = genomic DNA
gttgctgatcagctctgtgccaagtatagcaaggaatatggcaagctatgtaggaccaaccagattggaactgtgaatgacaggctaatgcacaagctgagtgtggaagccccacccaaaatcctggtggagagatacctgattgaaattgcaaagaattacaacgtaccctatgaacctgactctgtggtcatg

SEQ ID NO: 2 - DNA, 164 bp, Homo sapiens
  source 1..164: mol_type = genomic DNA
gcagtggcgttggtgggaagcgcagctcgaaaagcgatgccgattctggtttcctggggctgcggcccacttcggtggacccagcgctgaggcggcggcggcgaggcccaagaaataagaagcggggctggcggcggcttgctcaggagccgctggggctggag

SEQ ID NO: 3 - DNA, 22 bp, synthetic construct
  source 1..22: mol_type = other DNA
acgtaccctatgaacctgactc

SEQ ID NO: 4 - DNA, 20 bp, synthetic construct
  source 1..20: mol_type = other DNA
tggtcctacatagcttgcca

SEQ ID NO: 5 - DNA, 21 bp, synthetic construct
  source 1..21: mol_type = other DNA
cgaggcccaagaaataagaag

SEQ ID NO: 6 - DNA, 19 bp, synthetic construct
  source 1..19: mol_type = other DNA
agccccaggaaaccagaat

SEQ ID NO: 7 - DNA, 20 bp, synthetic construct
  source 1..20: mol_type = other DNA
agaaggctggggctcatttg

SEQ ID NO: 8 - DNA, 20 bp, synthetic construct
  source 1..20: mol_type = other DNA
aggggccatccacagtcttc

(19) United States
(12) Patent Application Publication
(10) Pub. No.: US 2024/0336968 A1
(43) Pub. Date: Oct. 10, 2024

(54) SERUM CIRCRNA MARKER FOR RECOGNIZING CRANIOSYNOSTOSIS AND APPLICATION THEREOF

(71) Applicant: NANJING MEDICAL UNIVERSITY, Nanjing, Jiangsu Province (CN)

(72) Inventors: Aihua GU, Nanjing (CN); Cheng XU, Nanjing (CN); Jin XU, Nanjing (CN); Zhenkun WENG, Nanjing (CN); Xin ZHANG, Nanjing (CN)

(73) Assignee: NANJING MEDICAL UNIVERSITY, Nanjing, Jiangsu Province (CN)

(21) Appl. No.: 18/293,068

(22) PCT Filed: Jul. 7, 2022
(86) PCT No.: PCT/CN2022/104367

§ 371 Date: Jan. 29, 2024

(30) Foreign Application Priority Data
Nov. 18, 2021 (CN) 202111371284.1

(51) Int. Cl. C12Q 1/6883; C12N 15/113

(52) U.S. Cl. CPC C12Q 1/6883; C12N 15/113; C12N 2310/532; C12Q 2600/158

(57) ABSTRACT

A serum circRNA marker for recognizing craniosynostosis and an application thereof. The marker is selected from one or more of hsa_circ_0003568 and hsa_circ_0000945. The marker combination has specificity and sensitivity to craniosynostosis, can be used for a kit for early diagnosis or monitoring of craniosynostosis, and provides a theoretical basis for the research of circRNA for craniosynostosis in the future.

### SEQUENCE LISTING

The present application contains a Sequence Listing that has been submitted electronically and is hereby incorporated by reference in its entirety. The electronic Sequence Listing is named Sequence_Listing_ST26, which was created on Jan. 23, 2024, and is 8,224 bytes in size.

### TECHNICAL FIELD

The present disclosure belongs to the fields of biotechnology and clinical medicine, and in particular relates to a serum circRNA marker for recognizing craniosynostosis and an application thereof.

### BACKGROUND

Craniosynostosis (CS) is a kind of birth defect of infant early-stage skull bone connection, is a kind of congenital craniofacial deformities caused by abnormal and non-physiologic suture fusion, and is considered to be a kind of undesirable development result. Craniosynostosis may lead to too early fusion of one or more sutura cranii, thus causing skull deformities and craniofacial asymmetry. It is estimated that one infant may have craniosynostosis in about every 2500 live-born infants. If it is not treated in time, CS may cause serious complications including special syndromes of intracranial hypertension symptoms, dysphrenia and dysnoesia, head-facial deformities, vision disorder and other special deformities. Therefore, the diagnosis and treatment of craniosynostosis are very important. As great importance is increasingly attached to molecular biology research at home and abroad, it would be desirable to disclose the occurrence and development of craniosynostosis from the molecular level and to have more accurate understanding on craniosynostosis at a molecular level. Early-stage definite diagnosis and effective intervention means implementation are the key of improving the survival and life quality of patients.

Circular RNA (circRNA) is a closed loop formed through reverse splicing by new kind of special non-coding RNA molecules discovered in recent years. Researches show that circRNA molecules play a role in cells in a form like “miRNA sponge” because they are rich in MicroRNA (miRNA) binding sites, and they can relieve the inhibition of the miRNA to its target genes. The circRNA molecules mainly participate in the regulation and control of gene expression, gene transcription and protein generation. More researches suggest that the abnormal expression of circRNA is closely related to the occurrence and development of diseases.

At present, the diagnostic criteria of craniosynostosis are mainly head shape abnormity and 3D skull CT reconstruction. However, due to high heterogeneity of craniosynostosis in clinics, it is difficult to be accurately diagnosed in early stage. Through researches in recent years, it is found that some specific circRNA has disease and tissue specificity, and due to its special closed loop configuration, circRNA is insensitive to nuclease, and thus has good stability, and this makes circRNA have an obvious advantage on the development and application as a clinical biomarker. The circRNA can exist stably for a long time in serum, and the loss of the circRNA may not be caused by various treatment methods such as RNA enzyme degradation, boiling, multigelation, acid or alkali environment and long-term preservation. Just because the circRNA has good stability and takes part in important physiological and pathological processes of bodies, it is very likely to be used as an important molecular marker and drug development target.

Through latest research results at home and abroad at present, it is discovered that there are hundreds kinds of circRNA in the serum, the expression quantity is abundant, the stability is relatively high, the quantitative detection is easy, and the specificity is higher. Researches show that the expression profile of serum circRNA may be used as a potential marker for early diagnosis of diseases such as colon cancer and lung cancer, but there are few reports about the research and development on circRNA associated with craniosynostosis. Therefore, it is of great significance on the early diagnosis of craniosynostosis to screen out the circRNA achieving craniosynostosis specificity abnormal expression and develop its diagnostic kit, and a new path is also opened for its drug screening, drug effect evaluation and targeted treatment.

### SUMMARY

The purpose of the present disclosure is to provide a serum circRNA marker for a craniosynostosis patient, and provides clinical support for early discovery and early treatment of craniosynostosis patients. The circRNA marker combination provided by the present disclosure has higher specificity and sensitivity.

Another purpose of the present disclosure is to provide an application of a reagent for detecting the serum circRNA marker.

A further another purpose of the present disclosure is to provide an auxiliary diagnosis reagent for craniosynostosis.

The present disclosure has the following specific implementation technical solution:

A serum circRNA marker associated with craniosynostosis is selected from any one or two of hsa_circ_0003568 and hsa_circ_0000945.

In the circRNA marker of the present disclosure, the nucleotide sequence of hsa_circ_0003568 is as shown by: GTTGCTGATCAGCTCTGTGCCAAGTATAGCAAG GAATATGGCAAGCTATGTAGGACCAACCAGATTGGAACTGTGAATGACAGGCTAA TGCACAAGCTGAGTGTGGAAGCCCCACCCAAAATCCTGGTGGAGAGATACCTGAT TGAAATTGCAAAGAATTACAACGTACCCTATGAACCTGACTCTGTGGTCATG (SEQ ID No.1); and
the nucleotide sequence of hsa_circ_0000945 is as shown by

(SEQ ID NO. 2)
GCAGTGGCGTTGGTGGGAAGCGCAGCTCGAAAAGCGATGCCGATTC
TGGTTTCCTGGGGCTGCGGCCCACTTCGGTGGACCCAGCGCTGAG
GCGGCGGCGGCGAGGCCCAAGAAATAAGAAGCGGGGCTGGCGGCG
GCTTGCTCAGGAGCCGCTGGGGCTGGAG.

Preferably, the circRNA marker is a combination of hsa_circ_0003568 and hsa_circ_0000945.

An application of a reagent for detecting the serum circRNA marker associated with craniosynostosis according to the present disclosure to preparation of an auxiliary diagnosis reagent for examining craniosynostosis is provided.

Preferably, the reagent for detecting the serum circRNA marker associated with craniosynostosis according to the present disclosure is a specific primer for detecting the circRNA marker.

More preferably, the sequences of a primer for detecting hsa_circ_0003568 are as follows:
hsa_circ 0003568 Forward: ACGTACCCTATGAACCTGACTC (SEQ ID NO.3), and
hsa circ 0003568 Reverse: TGGTCCTACATAGCTTGCCA (SEQ ID NO.4); and
the sequences of a primer for detecting hsa_circ_0000945 are as follows:
hsa_circ_0000945 Forward: CGAGGCCCAAGAAATAAGAA (SEQ ID NO.5); and
hsa_circ_0000945 Reverse: AGCCCCAGGAAACCAGAAT (SEQ ID NO.6).

The reagent for detecting the serum circRNA marker associated with craniosynostosis according to the present disclosure is a specific primer for detecting the circRNA marker of the present disclosure.

The sequences of a primer for detecting hsa_circ_0003568 are as follows: hsa_circ_0003568 Forward: ACGTACCCTATGAACCTGACTC (SEQ ID NO.3); and
hsa_circ_0003568Reverse: TGGTCCTACATAGCTTGCCA (SEQ ID NO.4); and
the sequences of a primer for detecting hsa_circ_0000945 are as follows:
hsa_circ 0000945 Forward: CGAGGCCCAAGAAATAAGAA (SEQ ID NO.5); and
hsa_circ_0000945 Reverse: AGCCCCAGGAAACCAGAAT (SEQ ID NO.6).

A kit for auxiliary diagnosis of craniosynostosis is provided and includes specific primers for detecting the circRNA marker of the present disclosure.

Preferably, the kit further includes a primer pair for detecting reference GAPDH genes, a reverse transcription reagent and a fluorescent quantitative PCR reaction solution, and specifically includes:
(1) forward and reverse primers for detecting GAPDH; and
(2) at least one of DNA polymerase, PCR buffer, MgCl₂, dNTPs, water and nucleic acid dye.

More preferably, the sequences of a primer pair for detecting reference GAPDH genes are as follows:
GAPDH Forward: AGAAGGCTGGGGCTCATTTG (SEQ ID NO.7), and
GAPDH Reverse: AGGGGCCATCCACAGTCTTC (SEQ ID NO.8).

The present disclosure provides a detection method for detecting the circRNA. The method specifically includes:
(1) extracting serum of peripheral blood: collecting 5 ml of fasting venous blood from each object of study with a vacuum anticoagulant EDTA blood collection tube, performing centrifugation by a standard method within 12 h, separating the serum into 1.5 ml centrifuge tubes in two shares, and performing cryopreservation at −80° C. for later use;
(2) extracting serum RNA: extracting the serum RNA with reference to a Trizol LS reagent operation process of Invitrogen Company, pretreating the serum by 12,000 rpm 4° C. centrifugation for 10 min in advance, preserving the obtained RNA at −80° C. for later use, and detecting the purity and concentration of the RNA by a NanoDrop ND-2000 instrument;
(3) screening out candidate circRNA through a Human circular RNA Array V2.0 chip, building a craniosynostosis serum circRNA expression profile, and screening out circRNA with differential expression, i.e., hsa_circ_0003568 and hsa_circ_0000945;
(4) performing qRT-PCR detection on circRNA: designing a circRNA specific reverse primer, performing RNA reverse transcription by using a reverse transcription kit produced by Takara Company to obtain cDNA, and then detecting the serum circRNA expression level by a SYBR-Green dye method (Real-time qPCR Master Mix, Takara, Japan) with a reaction system and conditions taking a reference to a kit manual; and
(5) quantifying hsa_circ_0003568 and hsa_circ_0000945 relative to the reference by a 2^{-ΔΔCt} method, and further analyzing and evaluating the expression of hsa_circ_0003568 and hsa_circ_0000945 in the serum on the early diagnosis value of craniosynostosis patients by an ROC (receiver operator characteristic curve) method.

The present disclosure has the beneficial effects that the present disclosure provides a serum circRNA marker associated with craniosynostosis. By drawing an ROC curve of serum hsa_circ_0003568 expression values and an ROC curve of hsa_circ_0000945 expression values of craniosynostosis patients and control population, and an ROC curve of serum hsa_circ_0003568 and hsa_circ_0000945 combination expression values of craniosynostosis patients and control population, the results show that the area under the hsa_circ_0003568 curve is 0.8329 (95% CI:0.7410-0.9248), the area under the hsa_circ_0000945 curve is 0.8372 (95% CI:0.7430-0.9314), and the area under the hsa_circ_0003568 and hsa_circ_0000945 combination curve is 0.9016 (95% CI:0.8289-0.9744). These results show that hsa_circ_0003568 and hsa_circ_0000945 can be determined as a strong diagnosis factor for diagnosing craniosynostosis. Therefore, the marker combination has specificity and sensitivity to craniosynostosis, and the detection reagent of this marker can be used for preparing a kit for early diagnosis or monitoring of craniosynostosis, provides a theoretical basis for the research of circRNA for craniosynostosis in the future, provides a novel concept for molecular level diagnosis and treatment of craniosynostosis, and has great theoretical significance and potential practical values.

### BRIEF DESCRIPTION OF THE DRAWINGS

FIG. 1: An ROC curve of serum hsa_circ_0003568 expression values of craniosynostosis patients and control population.

FIG. 2: An ROC curve of serum hsa_circ_0000945 expression values of craniosynostosis patients and control population.

FIG. 3: An ROC curve of serum hsa_circ_0003568 and hsa_circ_0000945 combination expression values of craniosynostosis patients and control population.

### DETAILED DESCRIPTION

The technical solution of specific implementations of the present disclosure will be described in detail below, the followings describe the exemplary embodiments of the present disclosure, and conditions and methods which are not specified in the implementations are all performed according to conventional conditions and methods. Test materials used in the following embodiments, unless otherwise specified, are all commercially available from routine biochemistry reagent shops.

### Example 1: Sample Collection

The inventor collected blood samples of craniosynostosis infants and normal controls conforming to the requirements from Nanjing Children's Hospital in July 2018 to October 2020. By data processing, 40 cases of healthy controls and 40 cases of craniosynostosis infants conforming to the requirements were selected as experimental subjects. Informed consent forms were signed by guardians of all research objects.

### Example 2: Extraction of Serum RNA

Operation was performed according to a Trizol LS (10296028) kit manual of Invitrogen Company. The specific steps were as follows:
(i) 750 uL of Trizol LS reagents were added into 300 uL of serum samples. (ii) The samples were uniformly mixed, and placed for 5 min at a room temperature (15 to 30° C.). (iii) 200 μl of chloroform was added, the materials were shaken vigorously for 30 s on a vortex oscillator, were placed on ice for 10 to 15 min, and were subjected to 12,000 rpm centrifugation for 10 min at 4° C., and supernatant was taken. (iv) Isopropanol having the same volume as the supernatant was added, and the materials are placed at a room temperature for 10 min. (v) 12,000 rpm centrifugation was performed for 15 min at 4° C., and supernatant was abandoned. (vi) 1 mL of 75% absolute ethyl alcohol was added, uniform mixing was performed, 7500 g centrifugation was performed for 10 min at 4° C., supernatant was abandoned, and washing was performed twice. (vii) RNA precipitates were dried at a room temperature to a transparent state, a proper amount of DEPC water was added, and the materials were placed for 2 to 3 hours at 4° C. so that the RNA could be sufficiently dissolved, the concentration was determined, and the materials were stored at −70° C.

### Example 3: Screening of circRNA Expression Profile

5 cases of craniosynostosis infants and 5 cases of normal controls were selected, Shanghai Kangcheng Biological Engineering Co., LTD was entrusted to build a circRNA expression profile by using a Human circular RNA Array V2.0 chip of Arraystar Company and screen out circRNA with differences, and then, samples were expanded for verification.

### Example 4: Preparation of cDNA Samples and Real-Time Quantitative PCR

(1) Preparation of a reverse reaction system as shown in Table 1:

TABLE 1

| Reagent | Volume | |
|---|---|---|
| 5× PrimeScript RT Master Mix | 2 | μL |
| Total RNA | χ | μL |
| RNase Free dH₂O | 10-χ | μL |

The χ reaction system could be correspondingly increased according to requirements, and the 10 μL reaction system may use 500 ng of Total RNA to the maximum degree. All operations were performed on ice.
(2) Reverse transcription:

Firstly, a reverse transcription reaction (37° C., 15 min) was performed, then, a reverse transcriptase inactivation reaction (85° C., 5 s) was performed, the temperature was lowered to 4° C. when the reaction was finished, and the sample was stored in a 4° C. refrigerator.

Used primers were designed and synthesized by Shanghai Invitrogen Biology Co., LTD, and the specific sequences were as shown in Table 2:

TABLE 2

| Name of primer | Sequence of primer (5′-3′) |
|---|---|
| hsa_circ_0003568 F | ACGTACCCTATGAACCTGACTC |
| | (SEQ ID NO. 3) |
| hsa_circ_0003568 R | TGGTCCTACATAGCTTGCCA |
| | (SEQ ID NO. 4) |
| hsa_circ_0000945 F | CGAGGCCCAAGAAATAAGAAG |
| | (SEQ ID NO. 5) |
| hsa_circ_0000945 R | AGCCCCAGGAAACCAGAAT |
| | (SEQ ID NO. 6) |
| H-GAPDH F | AGAAGGCTGGGGCTCATTTG |
| | (SEQ ID NO. 7) |
| H-GAPDH R | AGGGGCCATCCACAGTCTTC |
| | (SEQ ID NO. 8) |

(3) The expression level of circRNA in the sample was detected, and a used reaction system (10 uL) was as follows:

| | | |
|---|---|---|
| SYBR Green Master Mix | 5 | μL |
| Primer F | 0.5 | μL |
| Primer R | 0.5 | μL |
| cDNA | 2 | μL |
| ddH₂O | 2 | μL |

After the cDNA stock solution subjected to reverse transcription was diluted to 10 times, the PCR reaction system was added.

(4) Reaction conditions of qRT- PCR:

| | | | | |
|---|---|---|---|---|
| Predegeneration: | 95° | C. | 5 | min |
| Cycle reaction: | [95° | C. | 10 | s |
| 60° C. | 30 | s] | 40 | cycles |
| Solubility curve: | 95° | C. | 15 | s |
| 60° C. | 80 | s | | |
| 95° C. | 15 | s. | | |

A real-time fluorescent quantitative PCR reaction was performed by Roche RT-PCR LC480 II. After the reaction was finished, a solubility curve was analyzed on software, and a Ct value was calculated. GAPDH was used as a reference to correct a PCR cycle copy number. The differences of each gene between a treatment group and a control group were compared by calculating 2^{(-ΔΔ}^{^{Ct}}⁾.
(5) Experiment results:

Expression values of hsa_circ_0003568 in 40 cases of craniosynostosis patients and 40 cases of control population were as shown in Table 1. The expression level of the expression value of hsa_circ_0003568 in serum of 40 cases of craniosynostosis patients was obviously higher than that in serum of 40 cases of the control group (P=4.2×10⁻⁷). Expression values of hsa circ_0000945 in craniosynostosis patients and control population were as shown in Table 3. The expression level of the expression value of hsa_circ_0000945 in serum of craniosynostosis patients was obviously higher than that in serum of the control group (P=2.1×10⁻⁷).

TABLE 3
expression values and detection P values of serum
hsa_circ_0003568 and hsa_circ_0000945 of craniosynostosis
patients and control population in qRT - PCR detection

| | Control | Craniosynostosis | |
|---|---|---|---|
| | group | group | P value |
| has_circ_0003568 | 2.3 ± 1.1 | 3.8 ± 1.2 | 4.2 × 10⁻⁷ |
| has_circ_0000945 | 1.8 ± 0.6 | 3.0 ± 0.9 | 2.1 × 10⁻⁷ |

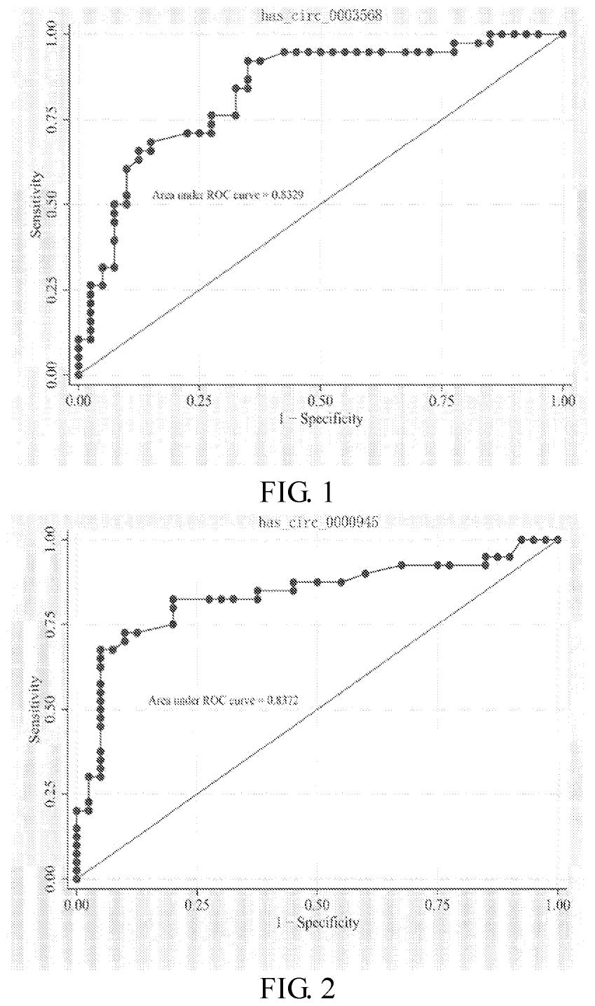

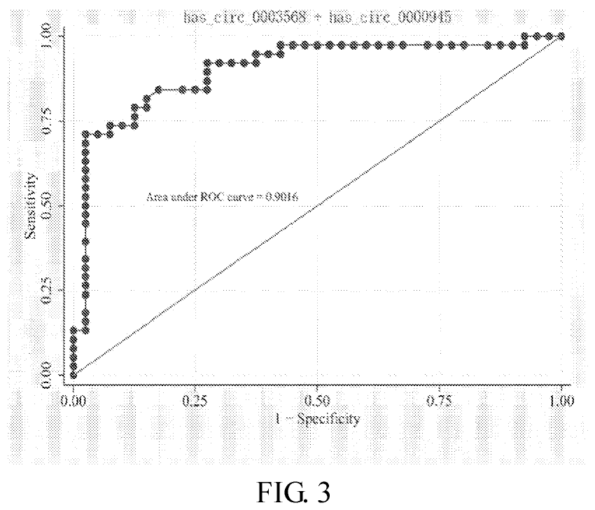

The marker expression was subjected to ROC analysis, and the diagnostic efficiency of the marker was judged. The ROC curve analysis results in FIG. 1 to FIG. 3 show that the area under hsa_circ_0003568 curve was 0.8329 (95% CI:0.7410-0.9248), the area under hsa circ 0000945 curve was 0.8372 (95% CI:0.7430-0.9314), and the area under hsa circ 0003568 and hsa_circ_0000945 combination curve was 0.9016 (95% CI:0.8289-0.9744). These results show that hsa_circ_0003568 and hsa_circ_0000945 can be determined as a strong diagnosis factor for diagnosing craniosynostosis. Additionally, the combination formed by hsa_circ_0003568 and hsa_circ_0000945 has a higher diagnosis value than single circRNA.

1. A serum circRNA marker associated with craniosynostosis, wherein the circRNA marker is selected from any one or two of hsa_circ_0003568 and hsa_circ_0000945, the nucleotide sequence of hsa_circ_0003568 is as shown in SEQ ID NO.1, and the nucleotide sequence of hsa_circ_0000945 is as shown in SEQ ID NO.2.

2. The serum circRNA marker associated with craniosynostosis according to claim 1, wherein the circRNA marker is a combination of hsa_circ_0003568 and hsa_circ_0000945.

3. An application of a reagent for detecting the serum circRNA marker associated with craniosynostosis according to claim 1 to preparation of an auxiliary diagnosis reagent for examining craniosynostosis.

4. The application according to claim 3, wherein the reagent for detecting the serum circRNA marker associated with craniosynostosis is a specific primer for detecting the serum circRNA marker associated with craniosynostosis.

5. The application according to claim 4, wherein the sequences of a primer for detecting hsa_circ_0003568 are as follows:
hsa_circ_0003568 Forward: SEQ ID NO.3, and
hsa_circ_0003568 Reverse: SEQ ID NO.4; and
the sequences of a primer for detecting hsa_circ_0000945 are as follows:
hsa_circ_0000945 Forward: SEQ ID NO.5, and
hsa_circ_0000945 Reverse: SEQ ID NO.6.

6. A reagent for detecting the serum circRNA marker associated with craniosynostosis according to claim 1, wherein the reagent is a specific primer for detecting the circRNA marker according to claim 1.

7. The reagent according to claim 6, wherein the sequences of a primer for detecting hsa_circ_0003568 are as follows:
hsa_circ_0003568 Forward: SEQ ID NO.3, and
hsa_circ_0003568 Reverse: SEQ ID NO.4; and
the sequences of a primer for detecting hsa_circ_0000945 are as follows:
hsa_circ_0000945 Forward: SEQ ID NO.5, and
hsa_circ_0000945 Reverse: SEQ ID NO.6.

8. A kit for auxiliary diagnosis of craniosynostosis, comprising any one of primers according to claim 6.

9. The kit according to claim 8, further comprising a primer pair for detecting reference GAPDH genes, a reverse transcription reagent and a fluorescent quantitative PCR reaction solution.

10. The kit according to claim 9, wherein the sequences of the primer pair for detecting the reference GAPDH genes are as follows:
GAPDH Forward: SEQ ID NO.7, and
GAPDH Reverse: SEQ ID NO.8.
